(19) United States
(12) Patent Application Publication
(10) Pub. No.: US 2004/0132051 A1
(43) Pub. Date: Jul. 8, 2004

(54) Mg-mediated hot start biochemical reactions

(76) Inventor: Mark R. Andersen, San Mateo, CA (US)

Correspondence Address: KILYK & BOWERSOX, P.L.L.C., 3603 CHAIN BRIDGE ROAD, SUITE E, FAIRFAX, VA, 22030, US

(21) Appl. No.: 10/625,436

(22) Filed: Jul. 23, 2003

Related U.S. Application Data
(60) Provisional application No. 60/398,934, filed on Jul. 26, 2002.

(51) Int. Cl. C12Q 1/68; C12M 1/34

(52) U.S. Cl. 435/6; 435/287.2

(57) ABSTRACT

Microfluidic devices are provided that are adapted to retain reactants in first and second chambers that can be in openable fluid communication with each other. The reactants can be reactants necessary to initiate, promote, or catalyze a polymerase chain reaction another nucleic acid sequence amplification, detection, ligation, or endonuclease reaction or a nucleic acid sequencing reaction. Methods and systems are also provided.

### CROSS-REFERENCE TO RELATED APPLICATION

This application claims the benefit under 35 U.S.C. § 119(e) of prior U.S. Provisional Patent Application No. 60/398,934, filed Jul. 26, 2002, which is incorporated herein in its entirety by reference.

### FIELD

The present teachings relate to nucleic acid detection, ligation, amplification, and sequencing reactions and devices to carry out such reactions.

### BACKGROUND

Various biochemical reactions advantageously occur or are advantageously facilitated at temperatures above normal room temperature. For this reason, reactants are added to a reaction chamber and heated to a suitable temperature. However, non-specific and/or undesired reactions can occur with reactants when the reactants are added to the reaction chamber under conditions that are other than ideal.

### SUMMARY

According to various embodiments, a microfluidic device is provided that includes a first chamber adapted to retain one or more first component for a desired reaction and a second chamber adapted to retain one or more second component for the desired reaction. The first and second chambers can be in openable fluid communication with each other. The first and second chambers can be adapted to retain components for a nucleic acid sequencing or nucleic acid sequence amplification reaction.

According to various embodiments, at least one reactant useful for initiating, catalyzing, promoting, or enzymatically activating a reaction is isolated from other reactants of a desired reaction until the reactant and the other reactants are heated to a elevated temperature. The temperature at which the reactants can be mixed can be sufficiently high so that base pairing of primers present as reactants cannot occur at locations with less than perfect or near-perfect homology.

A microfluidic device can include a first chamber and at least one first component retained in the first chamber, where the at least one first component can be one or more of a catalyst, an initiator, a promoter, and a cofactor for a desired reaction. The microfluidic device can include a second chamber and at least one second component retained in the second chamber. The at least one second component can include one or more reactant or reagent or component for the desired reaction. The microfluidic device can include an openable communication between the first and second chambers. A method is provided that can include the steps of providing the above-described microfluidic device, opening the openable fluid communication between the first and second chambers, and mixing the at least one first component with the at least one second component.

According to various embodiments, a microfluidic device can be provided that includes a first chamber that contains a magnesium catalyst, a second chamber that is capable of containing a magnesium-dependent enzyme and a target nucleic acid sequence, and an operable fluid communication between the first and second chambers. The openable fluid communication can be formed originally, for example, in a closed state.

### BRIEF DESCRIPTION OF THE DRAWINGS

FIG. 1 is a top plan view of a microfluidic device according to various embodiments;

FIG. 2 is a top plan view of a schematic drawing of a microfluidic device according to various embodiments;

FIG. 3A is a top view of a microfluidic device according to an embodiment wherein two recesses in a substrate are separated by an intermediate wall formed from a deformable inelastic material;

FIG. 3B is a cross-sectional side view of the assembly shown in FIG. 3A, taken along line 3B-3B of FIG. 3A;

FIG. 4A is a top view of the assembly shown in FIG. 3A along with a deformer device positioned after initiation of an intermediate wall deforming step;

FIG. 4B is a cross-sectional side view of the assembly and deformer shown in FIG. 4A, taken along line 4B-4B of FIG. 4A, and showing the contact surface of the deformer advancing toward the intermediate wall;

FIG. 5A is a top view of the assembly shown in FIG. 3A but wherein the intermediate wall is in a deformed state following contact of the deformer with the intermediate wall;

FIG. 5B is as cross-sectional side view of the assembly shown in FIG. 5A taken along line 5B-5B of FIG. 5A, showing the contact surface of the deformer retracting from the intermediate wall in a deformed state;

FIG. 6A is a partial cut-away top view of a substrate layer of the fluid manipulation valve assembly according to various embodiments, shown in an initial non-actuated stage;

FIG. 6B is a cross-sectional side view of the fluid manipulation valve assembly shown in FIG. 6A, taken along line 6B-6B as shown in FIG. 6A;

FIG. 7A is a top view of the substrate layer of the fluid manipulation valve assembly according to various embodiments, in a first stage of actuation of the valve assembly;

FIG. 7B is a cross-sectional side view of the fluid manipulation valve assembly shown in FIG. 7A, taken along line 7B-7B as shown in FIG. 7A, and corresponding to a first stage of actuation;

FIG. 8A is a top view of the substrate layer of the fluid manipulation valve assembly according to various embodiments, in a second stage of actuation of the valve assembly;

FIG. 8B is a cross-sectional side view of the fluid manipulation valve assembly shown in FIG. 8A, taken along line 8B-8B as shown in FIG. 8A, shown in a further deformed state corresponding to the second stage of actuation;

FIG. 9A is a top view of the substrate layer of the fluid manipulation valve assembly according to various embodiments, a third stage of actuation of the valve assembly;

FIG. 9B is a cross-sectional side view of the fluid manipulation valve assembly shown in FIG. 9A, taken along line 9B-9B as shown in FIG. 9A, corresponding to the third stage of actuation;

FIG. 10A is a top view of the substrate layer of the fluid manipulation valve prior to a fourth stage of actuation of the valve assembly;

FIG. 10B is a cross-sectional side view of the fluid manipulation valve assembly shown in FIG. 10A, taken along line 10B-10B as shown in FIG. 10A, and shown with the elastically deformable cover partially rebounded from the substrate layer;

FIG. 11A is a top view of the substrate layer of the fluid manipulation valve assembly according to various embodiments, shown without the elastically deformable cover and in a fourth stage of actuation of the valve assembly; and

FIG. 11B is a cross-sectional side view of the fluid manipulation valve assembly shown in FIG. 11A, taken along line 11B-11B as shown in FIG. 11A, and shown with the elastically deformable cover in a further deformed state, whereby the valve assembly has been re-closed corresponding to the fourth stage of actuation.

It is intended that the specification and examples be considered as exemplary only. The true scope and spirit of the present teachings includes various embodiments.

### DESCRIPTION OF VARIOUS EMBODIMENTS

According to various embodiments, the terms “polynucleotide,” “DNA,” and “DNA fragments” as used herein, can include nucleic acid analogs that can be used in addition to or instead of nucleic acids. Examples of nucleic acid analogs include the family of peptide nucleic acids (PNA), wherein the sugar/phosphate backbone of DNA or RNA has been replaced with acyclic, achiral, and neutral polyamide linkages. For example, a probe or primer can have a PNA polymer instead of a DNA polymer. The 2-aminoethylglycine polyamide linkage with nucleobases attached to the linkage through an amide bond has been well-studied as an embodiment of PNA and shown to possess exceptional hybridization specificity and affinity. An example of a PNA is as shown below in a partial structure with a carboxyl-terminal amide:

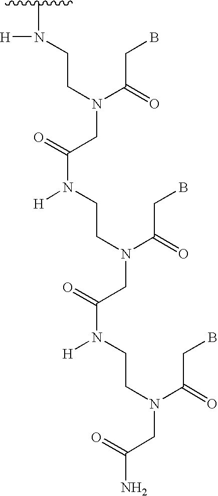

“Nucleobase” as used herein means any nitrogen-containing heterocyclic moiety capable of forming Watson-Crick hydrogen bonds in pairing with a complementary nucleobase or nucleobase analog, e.g. a purine, a 7-deazapurine, or a pyrimidine. Typical nucleobases are the naturally occurring nucleobases such as, for example, adenine, guanine, cytosine, uracil, thymine, and analogs of the naturally occurring nucleobases, e.g. 7-deazaadenine, 7-deazaguanine, 7-deaza-8-azaguanine, 7-deaza-8-azaadenine, inosine, nebularine, nitropyrrole, nitroindole, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-azapurine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, 4-methylindole, pyrazolo[3,4-D]pyrimidines, “PPG”, and ethenoadenine.

“Nucleoside” as used herein refers to a compound consisting of a nucleobase linked to the C-1′ carbon of a sugar, such as, for example, ribose, arabinose, xylose, and pyranose, in the natural β or the α anomeric configuration. The sugar can be substituted or unsubstituted. Substituted ribose sugars can include, but are not limited to, those riboses having one or more of the carbon atoms, for example, the 2′-carbon atom, substituted with one or more of the same or different Cl, F, —R, —OR, —NR₂ or halogen groups, where each R is independently H, C₁-C₆ alkyl or C₅-C₁₄ aryl. Ribose examples can include ribose, 2′-deoxyribose, 2′,3′-dideoxyribose, 2′-haloribose, 2′-fluororibose, 2′-chlororibose, and 2′-alkylribose, e.g. 2′-O-methyl, 4′-α-anomeric nucleotides, 1′-α-anomeric nucleotides, 2′-4′- and 3′-4′-linked and other “locked” or “LNA”, bicyclic sugar modifications. Exemplary LNA sugar analogs within a polynucleotide can include the following structures:

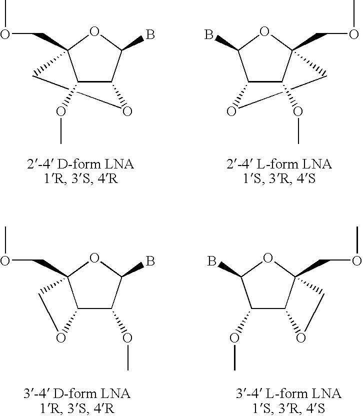

where B is any nucleobase.

Sugars can have modifications at the 2′- or 3′-position such as methoxy, ethoxy, allyloxy, isopropoxy, butoxy, isobutoxy, methoxyethyl, alkoxy, phenoxy, azido, amino, alkylamino, fluoro, chloro and bromo. Nucleosides and nucleotides can have the natural D configurational isomer (D-form) or the L configurational isomer (L-form). When the nucleobase is a purine, e.g. adenine or guanine, the ribose sugar is attached to the N⁹-position of the nucleobase. When the nucleobase is a pyrimidine, e.g. cytosine, uracil, or thymine, the pentose sugar is attached to the N¹-position of the nucleobase.

“Nucleotide” as used herein refers to a phosphate ester of a nucleoside and can be in the form of a monomer unit or within a nucleic acid. “Nucleotide 5′-triphosphate” as used herein refers to a nucleotide with a triphosphate ester group at the 5′ position, and can be denoted as “NTP”, or “dNTP” and “ddNTP” to particularly point out the structural features of the ribose sugar. The triphosphate ester group can include sulfur substitutions for the various oxygens, e.g. α-thio-nucleotide 5′-triphosphates.

As used herein, the terms “polynucleotide” and “oligonucleotide” mean single-stranded and double-stranded polymers of, for example, nucleotide monomers, including 2′-deoxyribonucleotides (DNA) and ribonucleotides (RNA) linked by internucleotide phosphodiester bond linkages, e.g. 3′-5′ and 2′-5′, inverted linkages, e.g. 3′-3′ and 5′-5′, branched structures, or internucleotide analogs. Polynucleotides can have associated counter ions, such as H⁺, NH₄⁺, trialkylammonium, Mg²⁺, Na⁺ and the like. A polynucleotide can be composed entirely of deoxyribonucleotides, entirely of ribonucleotides, or chimeric mixtures thereof. Polynucleotides can be comprised of internucleotide, nucleobase and sugar analogs. For example, a polynucleotide or oligonucleotide can be a PNA polymer. Polynucleotides can range in size from a few monomeric units, e.g. 5-40 when they are more commonly frequently referred to in the art as oligonucleotides, to several thousands of monomeric nucleotide units. Unless otherwise denoted, whenever a polynucleotide sequence is represented, it will be understood that the nucleotides are in 5′ to 3′ order from left to right and that “A” denotes deoxyadenosine, “C” denotes deoxycytidine, “G” denotes deoxyguanosine, and “T” denotes thymidine, unless otherwise noted.

“Internucleotide analog” as used herein means a phosphate ester analog or a non-phosphate analog of a polynucleotide. Phosphate ester analogs can include: (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate. Non-phosphate analogs can include compounds wherein the sugar/phosphate moieties are replaced by an amide linkage, such as a 2-aminoethylglycine unit, commonly referred to as PNA.

According to various embodiments, an activating agent or component can be preloaded into one of the first chamber and the second chamber. The activating component can be or can include one or more of a catalyst, an initiator, a promoter, a cofactor, or an enzyme, for example. If the activating component includes magnesium, the magnesium can be in the form of a salt, for example, magnesium chloride (MgCl₂), magnesium sulfate (MgSO₄), magnesium acetate (Mg(OAc)), or combinations thereof The magnesium can be in an aqueous solution containing Mg²⁺ ions. The magnesium can be hydrated ((Cl₃Co₂)₂Mg). The magnesium can be a magnesium salt dried down and deposited in one of the first or second chambers or can be dried down in one of the first and second chambers. Magnesium can be contained in a substance in a form such that divalent magnesium can be released into an aqueous solvent. A magnesium compound can have a concentration of, for example, from about 1 mM to about 5 mM at 1× or up to 500 mM at, for example, 100×. The final concentration of the mixture of the first and second chambers can be, for example, from about 1 mM to about 5 mM. For example, the substance can release magnesium into an aqueous solvent having a pH of from about 6 to about 9 when the mixture of the substance and the solvent is heated to a temperature from about 50° C. to about 100° C. for an interval of from about 0.5 to about 5 minutes. An exemplary heating device that can be used to heat the microfluidic device, is described in U.S. patent application Ser. No. 10/359,668, filed Feb. 6, 2003, which is incorporated herein in its entirety by reference. A magnesium solution can be preloaded into the second chamber and can be dried down prior to the first and second chambers being in fluid communication.

According to various embodiments, the one of the first chamber and the second chamber can retain various components or reagents for performing a polymerase chain reaction. The contents of the other chambers can optionally not contain other, various components or reagents for performing a polymerase chain reaction. For example, the second chamber can retain an activating agent as described herein, and the first chamber can retain at least one buffer, a polymerase, dNTPs, and at least one primer. The activating agent can be, for example, a magnesium catalyst. The components and/or reagents retained in the first chamber can be mixed, dissolved, or contained in an aqueous solution. The polymerase can be, for example, a thermostable enzyme such as thermos aquatus (Taq polymerase). The aqueous solution can have a volume of from about 0.02 to about 200 μl. The aqueous solution can be a buffer. The buffer can have a pH of, for example, from about pH 8 to about pH 9 at room temperature. The aqueous buffer can contain, for example, about 0.05M potassium chloride (KCl). The dNTPs, for example, dATP, dTTP, dCTP, and dGTP, can have a concentration of, for example, from about 50 μM to about 100 μM. The primers can be oligonucleotide primers, such as single-stranded DNA primers, single-stranded LNA primers, or single-stranded chimeric PNA primers “doped primers.” The primers can be up to, for example, 15, 30, 45, 60, or more nucleotides long and can contain base sequences that are Watson-Crick complementary to sequences on one or both strands of the target nucleic acid sequences. The primers can be present at a concentration of, for example, from about 50 to about 2000 nanomolars. To perform a sequencing reaction, at least some of the dNTPs can be ddNTPs, or dideoxynucleotide triphosphates.

According to various embodiments, at least one of the first and second chambers can retain or contain one or more activating agents for an isothermal nucleic acid sequence amplification or sequencing reaction.

According to various embodiments, at least one of the first or second chambers can retain or contain components necessary to perform a ligase chain reaction (LCR), an oligonucleotide ligase assay (OLA), a ligase assay (LA), or an endonuclease reaction. The contents of the other chamber can optionally not contain other, various components or reagents for performing at least one of the above-mentioned reactions and/or assays. For example, the second chamber can retain an activating agent as described herein, and the first chamber can retain at least one buffer, dNTPs, and at least one probe. The first chamber can retain a ligase, an endonuclease, or other enzyme. The components and/or reagents retained in the first chamber can be mixed, dissolved, or contained in an aqueous solution. The enzymes can be thermostable enzymes. The enzymes can be magnesium-dependent or magnesium-mediated enzymes. The second chamber can retain or contain magnesium.

According to various embodiments, at least one first chamber can retain various components or reagents for performing a ligation reaction, a second chamber can retain a solution including magnesium, a salt of magnesium, or a solution containing magnesium, and a third chamber can retain various components or reagents for a polymerase chain reaction. The at least one first chamber, the second chamber, and the third chamber, can be in openable fluid communication with at least one of the other chambers. The contents of the second chamber can be heated and an openable communication between the second chamber and the first chamber can be opened to form a fluid communication between the at least one first chamber and the second chamber and the contents of the at least one first chamber and the second chamber can be combined and/or mixed. The contents of the first and second chambers can be cooled, heated, or both, and the first and second chambers, and the third chamber can be made to be in fluid communication with the mixed contents of the at least one first chamber and the second chamber. The admixed contents can be further combined and/or mixed with another chamber containing magnesium, a salt of magnesium, or a solution containing magnesium. The contents can be heated, cooled, or both, prior to the further combining and/or mixing.

According to various embodiments, the second chamber can retain various components or reagents for performing a magnesium-dependent or magnesium-mediated enzymatic reaction. For example, magnesium, a salt of magnesium, or a solution containing magnesium, can be retained in the second chamber.

According to various embodiments, the microfluidic device can be of the size, shape and general layout of a compact disk (CD). According to various embodiments, the microfluidic device can be a card, for example, a rectangular microfluidic device card. The card can have one or more notch or other feature that orients the card in another device, for example, in a card holder or on a rotating platen. The microfluidic device can be adapted to fit into a microfluidic device holder or rotating platen. The platen can be attached to or connected with a mechanical device to spin the microfluidic device, heat the microfluidic device, agitate the microfluidic device, move the microfluidic device, or perform other physical manipulations of the microfluidic device, or combinations thereof.

According to various embodiments, the microfluidic device can be a monolithic structure. The microfluidic device can have at least two chambers adapted to retain solutions or other reagents. The microfluidic device can have one or more valves that can be adapted to place at least two chambers in fluid communication. The microfluidic device can have a first side and a second side. Valves, chambers, fluid passages, or combinations thereof, can be located on the first side, the second side, or both sides of the microfluidic device. Valves or fluid passages can connect chambers on the first side of microfluidic device with chambers on the second side of the microfluidic device. The chambers, valves, or fluid passages can have at least one side wall. The chambers can be adapted to retain, contain, receive, restrain, archive, hold, and/or dispense reagents. The chambers can be adapted to retain reactants during chemical reactions, for example, a polymerase chain reaction, a ligase chain reaction, an oligonucleotide ligase assay, an endonuclease assay, or a nucleic acid sequencing reaction. The chambers can be adapted to perform filtration or purification of reagents or solutions.

One or more cover layers can cover the first and/or second sides of the microfluidic device. The cover layer can be optically clear. The cover layer can be thermally conductive. The cover layer can be elastically deformable or semi-elastically deformable. Adjacent sections of the cover layer can be made of one or more different materials.

Examples of microfluidic device features and systems for spinning, heating, cooling, and otherwise processing microfluidic devices, that can be useful in or with the microfluidic devices described herein, are described, for example, in U.S. patent applications Ser. Nos. 10/336,274, 60/398,851, 10/336,274, 60/399,548, 10/336,706, 60/398,777, 10/403,652, 60/398,946, 10/336,330, and 10/403,640, which are incorporated herein in their entireties by reference.

According to various embodiments, the chambers can be preloaded with reagents or reactants. For example, the first or second chamber can be preloaded with an aqueous solution containing magnesium ions. The first or second chamber can be preloaded with a buffer solution, Taq polymerase, a ligase, an endonuclease, dNTPs, one or more salt, such as KCl, one or more primer, one or more probe, or combinations thereof A user can load a sample containing DNA into a third chamber that can be in fluid communication with the first and/or second chambers by one or more valves. The first chamber can contain the necessary components of a single-tube assay, described, for example, in PCT International Patent Application No. PCT/US 03/02238, filed on Jan. 27, 2003, which is incorporated herein in its entirety by reference.

According to various embodiments, the valve can be a pressure-sensitive one-way valve or a single use valve. The valve can be an inelastically deformable barrier. For example, the valve can be a deformable barrier where one or more sidewalls of the valve can be deformed to close the valve. Alternatively, or additionally, a barrier can be deformed to open the valve. The valve can be a Zbig valve. The valve can include an elastic material. The valve can be as described for example, in U.S. patent applications Ser. Nos. 60/398,851, 10/336,274, 60/399,548, 10/336,706, 60/398,777, 10/403,652, 60/398,946, 10/336,330, and 10/403,640, which are incorporated herein in their entireties by reference.

One of the first and second chambers can be loaded with one or more reactants necessary to perform a nucleic acid polymerase chain reaction. The first and/or second chamber can include buffers, salts, polymerases, other enzymes, dNTPs, primers, and sample nucleic acid sequence, DNA, or DNA fragment. According to various embodiments, the contents of the chamber with such components can be heated to a temperature sufficient to denature a majority of the sample. For example, the temperature can rapidly be heated from room temperature to from about 90° C. to about 100° C. According to various embodiments, a valve, for example, an inelastically deformable valve between the first and second chambers, can be opened while the contents of one of the first and second chambers are heated, so that the first and second chambers become in fluid communication with each other. The microfluidic device, for example, a microfluidic card device, containing the first and second chambers, can be mounted on a platen that is connected to a mechanical device. The platen can be rotated to move the contents of the first chamber into the second chamber using centripetal force. While rotating or spinning, heat can be continued to be applied to the first and second chambers. After the contents of the first and second chambers have been mixed in the second chamber, the temperature of the second chamber containing the reaction mixture can be lowered to from about 50° C. to about 65° C. When the temperature of the second reaction chamber is from about 50° C. to about 65° C., a first cycle of sample nucleic acid sequence amplification can occur.

According to various embodiments, at least one of the chambers can be pre-heated before the contents of the first and second chambers are combined and/or mixed. The chambers can be pre-heated to a temperature less than from about 90° C. to about 100° C. The first and/or second chambers can be heated before, during, or after the contents of the chambers are combined and/or mixed. For example, the first and second chambers can be pre-heated prior to combining the contents of the first and second chambers. After the contents of the first and second chambers are combined, the chamber containing the combined contents can be heated to a temperature of from about 90° C. to about 100° C. For example, the contents of the first and second chambers can be combined when at room temperature and shortly after the contents are combined, the chamber containing the combined contents can be heated to a temperature of from about 90° C. to about 100° C.

According to various embodiments, the contents of the first and second chambers can be combined and mixed at different times or at the same time. For example, the contents of the first chamber can be combined with the contents of the second chamber using centripetal force by spinning a platent containing the first and second chambers at a low rate, or rounds per minute (RPM), for example, from about 100 RPM to about 1,000 RPM. The contents of the first and second chambers can be mixed by centripetal force by spinning a platent containing the first and second chambers at a relatively high RPM, for example, from about 2,500 RPM to about 5,000 RPM. The contents of the first and second chambers can be combined using centripetal force, a positive pressure gradient, for example, a positive pressure gradient created by heat, or a negative pressure gradient, for example a negative pressure gradient created by a vacuum. The contents of the first and second chambers can be mixed by, for example, centripetal force, thermal mixing, vortexing, shaking, sonication, or thermally-activated solutization. The contents of the first and second chambers can have different viscosities. For example, an aqueous solution containing magnesium can be mixed with glycerol and preloaded into the second chamber. The assay reactants contained in the first chamber can be slowly transferred into the second chamber to create two separate layers. The second chamber can then be heated to mix the first and second layers containing the assay reactants and the magnesium, respectively.

According to various embodiments, a method is provided that can include providing a device according to various embodiments described herein, opening the openable fluid communication, and mixing the contents of at least a first and a second chamber. The method can include preloading at least one of the first and second chambers with a catalyst for a desired reaction, for example, a magnesium or a magnesium catalyst-containing solution useful for nucleic acid sequence amplification and/or sequencing reaction. The method can include loading a sample containing DNA, and other PCR reactants, with the exception of magnesium or magnesium catalyst-containing solution, into the first chamber. The method can include loading a PCR master mix into the first chamber. The method can include loading a single tube assay, available from Applied Biosystems, Foster City, Calif., a sample containing DNA, or combinations thereof, into the first chamber. The method can include loading one or more ligase or ligase enzyme into the first chamber. The method can include loading a sample containing DNA, DNA fragments, amino acids, and/or a combination thereof, into the first chamber. The method can include loading at least one enzyme into the first chamber. The method can include loading at least one catalyst into the first chamber. The method can include loading or preloading at least one of the first or second chambers with one or more components needed for at least one chemical reaction. The method can include loading or preloading one or more chambers in a device having multiple chambers, with one or more components needed for at least one chemical reaction.

According to various embodiments, a method is provided that can include methods to amplify DNA. Methods to amplify DNA can include, for example, amplifying DNA by polymerase chain reaction, assays and reagents available from Applied Biosystems, Foster City, Calif., and flap-endonuclease amplification, assays and reagents available from Third Wave Technologies, Inc. Madison, Wis. According to various embodiments, the method can also include, for example, methods of hybridizing DNA and DNA fragments and methods of ligating DNA and DNA fragments.

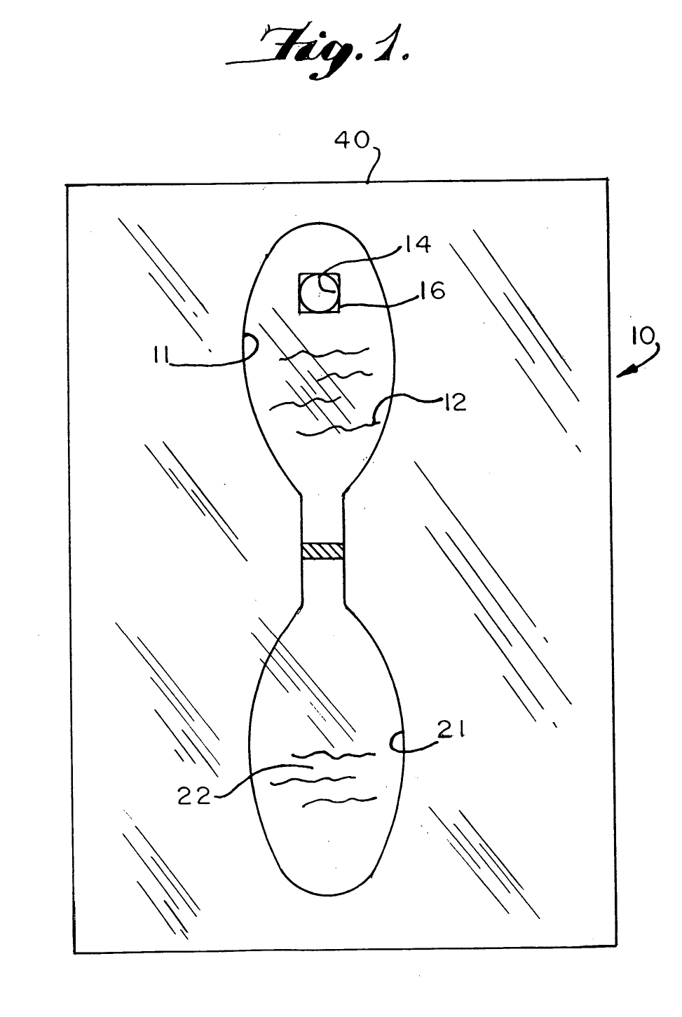

FIG. 1 is a top plan view of a microfluidic device 10. The microfluidic devices includes a first chamber 11 and a second chamber 21. Assay reactants 12, containing assay reactants for performing a nucleic acid sequence amplification reaction, including a target DNA sequence but excluding an activating agent such as magnesium ions, are retained within first chamber 11 in an aqueous solution 12. The second chamber 21 retains one or more activating agent 22 for activating a reaction of the assay reactants. For example, the second chamber 21 can contain a magnesium ion catalyst solution. Chambers 11 and 21 can be isolated from the atmosphere by a cover layer 40, for example, a polyolefin film layer and/or an optically clear cover layer. First chamber 11 and second chamber 21 can be separated by a deformable intermediate wall 30. Optically clear cover layer 40 can be an elastically deformable plastic that overlays the top of the microfluidic device 10. Cover layer 40 can be attached to the microfluidic device, for example, with an adhesive, by heat bonding, and/or by ultrasonic welding, for example. In the embodiment shown, the first chamber 11 can include an entrance port 14 that can be sealed by an adhesive sealing tape 16. Other suitable sample introduction openings, apertures, vents, holes, or the like, can be included with the microfluidic device.

The deformable intermediate wall 30 can be deformed by an opening device (not shown), to place first chamber 11 in fluid communication with second chamber 21. The assay reactants can be combined with a solution containing activating agent 22 by moving the assay reactants 12 into the second chamber 21. The assay reactants can be moved using a positive pressure gradient, a negative pressure gradient, centripetal force, or combinations thereof. A larger pressure gradient can be applied over a shorter period of time or a smaller pressure gradient can be applied over a longer period of time. A larger centripetal force can be applied over a shorter period of time or a smaller centripetal force can be applied over a longer period of time. The second chamber can be heated before, during, or after the assay reactants and the activating agent 22 are combined. One or more of the first and second chambers can be preheated prior to combining the assay reactants 12 and the activating agent 22. The assay reactants 12 and the activating agent 22 can be mixed during or after combination. The assay reactants 12 and the activating agent 22 can be mixed using pressure gradients, centripetal force, thermal mixings, vortexing, shaking, or the like.

FIG. 2 is a top plan view of a microfluidic device 50 having a plurality of series of chambers 60, 62, and 64. Various assay reactants, reagents, activatable components, and activating agents (not shown) can be placed in any of chambers 60, 62, and 64. A ligase and a sample containing a target nucleic acid sequence, along with other associated reagents, can be placed, for example, in the chambers 64. A magnesium salt, such as, for example, MgCl₂, can be placed in the chambers 62. PCR components, including, for example, primers and probes, can be placed in the chambers 60. The microfluidic device 50 can be manipulated with a system (not shown) and the temperature of the chambers can be elevated to a temperature sufficient to efficiently cause, motivate, promote, maintain, or continue a biochemical reaction of the ligase with the target nucleic acid sequence when in the present of the magnesium.

Chambers 64 and 62 can then be made to be in fluid communication with each other, without exposing the contents of chambers 64 and 62 to possible contamination. Chambers 64 and 62 can be fluidly communicated, for example, by inelastically deforming a deformable barrier 63 between chambers 64 and 62. The contents of chambers 64 and 62 can then be mixed using centripetal force by rotating the microfluidic device 50 to cause a radially outward flow of components from chamber 64 to chamber 62. After performing the ligase reaction, chambers 62 and 60 can be placed in fluid communication with each other by deforming barrier 61.

The contents of chambers 60, 62, and 64 can be combined and/or mixed using centripetal force by rotating the microfluidic device 50, to initiate a reaction of the various components, for example, a polymerase chain reaction. The contents of the chambers 60, 62, and 64 can be cooled, heated, or combinations thereof, between the ligase reaction and the polymerase chain reaction. For example, the temperature level can be permissive to initiate, promote, maintain, or activate a chemical reaction for use with real-time monitoring of a polymerase chain reaction. Room temperature can be sufficient to initiate, promote, or maintain a flap endonuclease (FEN) reaction according to various embodiments.

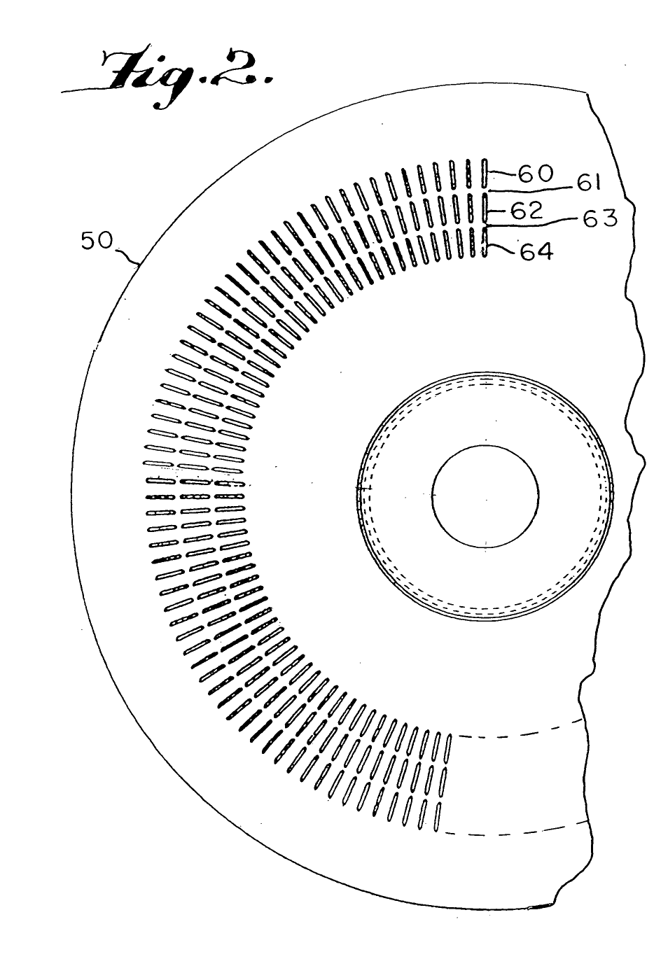

FIG. 3A is a top view of a microfluidic assembly 198 according to an embodiment wherein two chambers to be initially kept separate, in the form of recesses 106 and 107, are formed in a substrate layer 100 and are separated by an intermediate wall 108 formed from a deformable material. The material of the intermediate wall can be inelastically deformable or elastically deformable.

If the material of the intermediate wall is elastically deformable, it can be less elastically deformable (have less elasticity) than the material of the cover layer, or at least not as quickly elastically rebounding as the material of the cover layer, whereby the cover layer is able to recover or rebound from deformation, more quickly than the intermediate wall material. Thus, if both the cover layer and the intermediate wall are elastically deformable but to different degrees, the cover layer can rebound from deformation more quickly than the intermediate wall material and a gap can therefore be provided therebetween, that can function as an opening for a fluid communication. For the sake of example, but not to be limiting, the intermediate wall material will be described below as being inelastically deformable.

FIG. 3B is a cross-sectional side view of the assembly 198 shown in FIG. 3A, taken along line 3B-3B of FIG. 3A. The assembly 198 also includes an elastically deformable cover layer 104 and a pressure-sensitive adhesive layer 102 disposed between the substrate 100 and the elastically deformable cover layer 104. The recess 106 is at least partially defined by sidewalls 116 and 118 and bottom wall 114 as shown in FIG. 3B. In the non-deformed state, intermediate wall 118 has a top surface that is in contact with and sealed by the pressure sensitive adhesive 102 at interface 103.

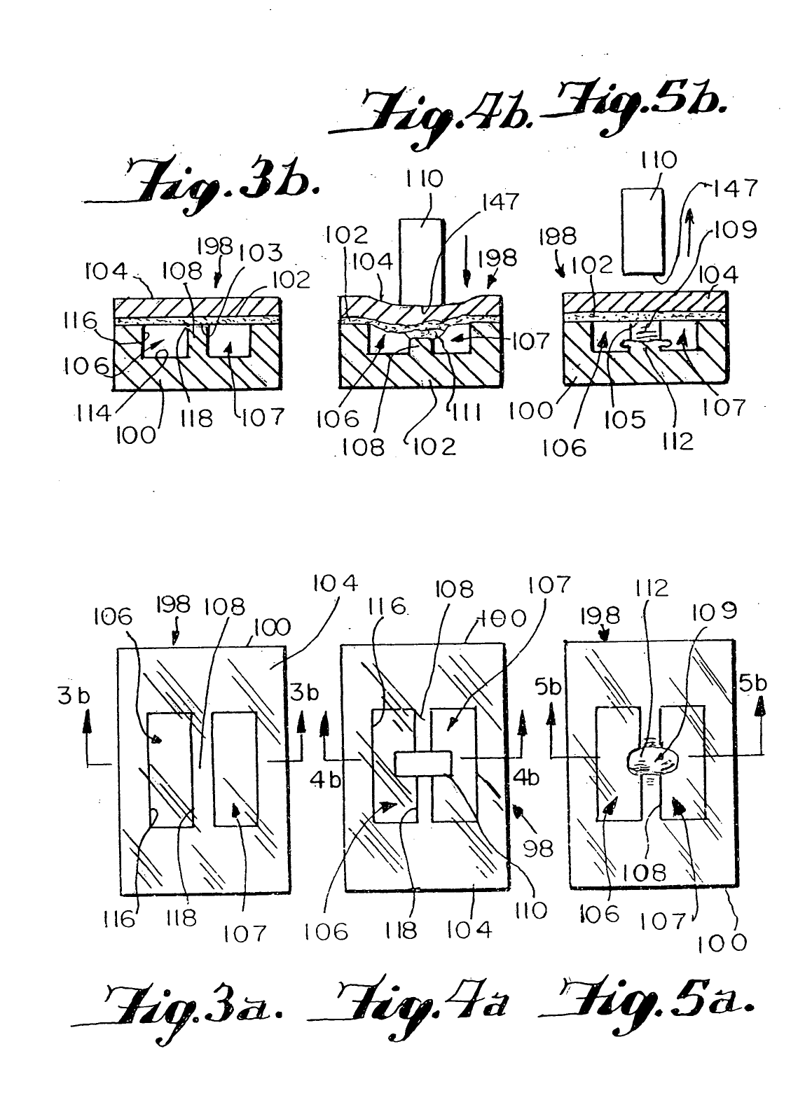

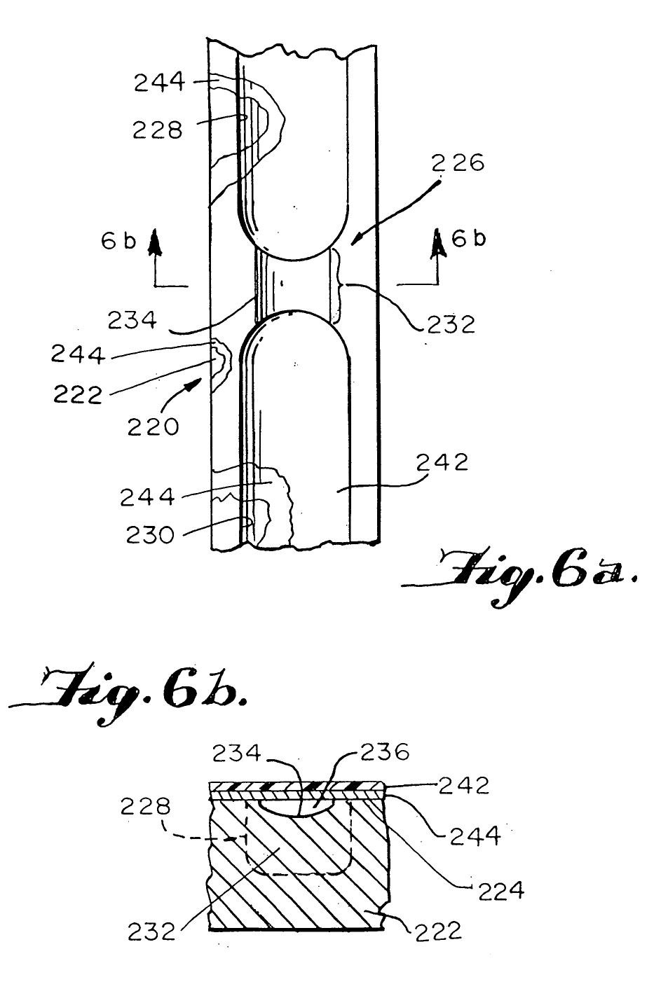

FIG. 4A is a top view of the assembly 198 shown in FIG. 3A in deforming contact with a deformer 110 positioned after initiation of and during an intermediate wall-deforming step. FIG. 4B is a cross-sectional side view of the assembly 198 and deformer 110 shown in FIG. 4A, taken along line 4B-4B of FIG. 4A, and showing the contact surface 147 of the deformer 110 advancing toward and deforming the intermediate wall 108. FIG. 5A is a top view of the assembly shown in FIG. 3A but wherein the intermediate wall is in a deformed state following contact of the deformer with the intermediate wall. FIG. 5B is a cross-sectional side view of the assembly 198 shown in FIG. 5A with the deformer 110, with the assembly 198 being taken along line 5B-5B of FIG. 5A. FIG. 5B shows the contact surface of the deformer 110 retracting from the intermediate wall 108 leaving a portion 112 in a deformed state.

As can be seen in FIG. 4B, the deformer 110 deforms the cover layer 104, the pressure sensitive adhesive layer 102, and the intermediate wall 108. The intermediate wall 108 gives way to the deforming force of the deformer and begins to bulge as shown at 111. After the deformer 110 is withdrawn from contact from the assembly 198, the elastically deformable cover layer 104 and pressure sensitive adhesive layer 102 rebound to return to their original orientation, however, the inelastically deformable material of the intermediate wall 108 remains deformed after withdrawal of the deforming force such that intermediate wall 108 is provided with a depressed, deformed portion 112. The portion of the elastically deformable cover layer 104, including the pressure sensitive adhesive layer 102, adjacent the deformed portion 112 of the intermediate wall 108, is not in contact with the deformed portion 112 such that a through-passage 109 is formed allowing fluid communication between recesses 106 and 107.

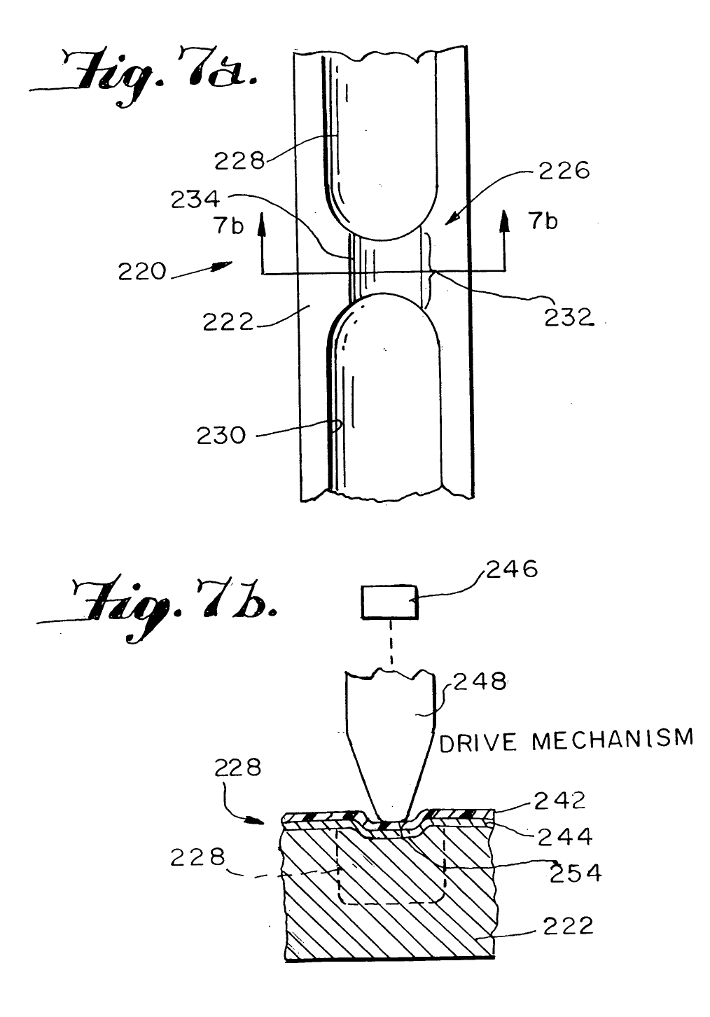

FIG. 6A shows a partial cut-away top view of a substrate layer portion 222 of a fluid manipulation valve assembly 220 according to various embodiments. At least two recesses 228, 230 can be formed in the substrate layer 222, and can be separated by an intermediate wall 232. The intermediate wall 232 can define an area of a valve 226 that can be manipulated to control fluid communication between the two recesses 228, 230. The intermediate wall 232 can be formed from a deformable material that can be inelastically or elastically deformable. According to various embodiments, the entire substrate layer 222 can include an inelastically or elastically deformable material.

FIG. 6B is a cross-sectional side view of the valve 226 shown in FIG. 6A, taken along line 6B-6B of FIG. 6A. The valve 226 can include an elastically deformable cover including a cover layer 242 and an adhesive layer 244. The adhesive layer 244 can include, for example, a pressure sensitive or hot melt adhesive, disposed between the substrate layer 222 and the elastically deformable cover layer 242.

As shown in FIG. 6B, a height of the intermediate wall 232 between the recesses 228, 230 can be formed with a depression relative to a surface 224 of the substrate layer 222, thereby forming a recessed channel 234. Moreover, the non-depressed portion of the intermediate wall 232 can be flush with a top surface 224 of the recess-containing substrate layer 222 of the assembly 220. As illustrated in FIG. 6B, in the non-deformed state of the cover layer 242, the recessed channel 234 of the intermediate wall 232 can form a fluid communication 236 between the first recess 228 and the second recess 230. Therefore, in the non-deformed state of the elastically deformable cover, the valve 226 is in a normally open condition. According to various embodiments, the valve 226 of the fluid manipulation valve assembly 220 can be manipulated using mechanical pressure, and temperature, for example.

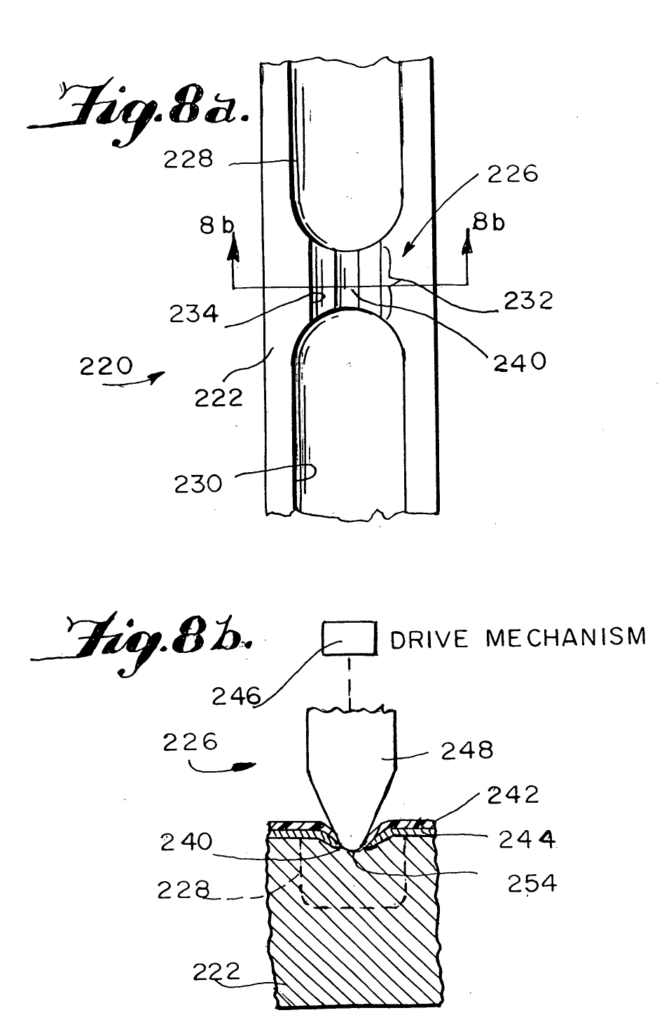

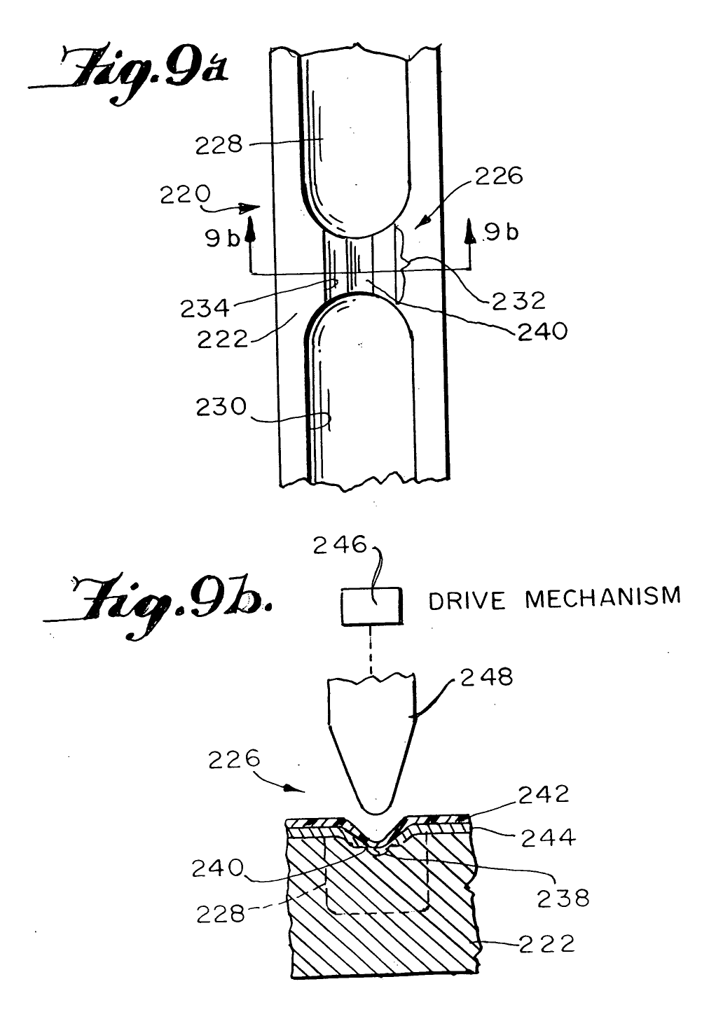

FIGS. 7A and 7B show a top view and a cross-sectional side view, respectively, of the valve 226 of the fluid manipulation valve assembly 220 in the first valve closing condition. In FIG. 7B, the valve 226 is shown in deforming contact with a first deformer 248 positioned after initiation of, and during, the first valve closing condition. As can be seen in FIG. 7B, a drive mechanism 246 can be arranged to displace the first deformer 248 in a direction towards the cover layer 242 such that a contact surface 254 of the first deformer 248 deforms the cover layer 242 and the adhesive layer 244 towards the recessed channel 234. FIG. 7A illustrates a top view of the substrate layer portion 222 when the valve 226 is in the first valve closing condition. In FIG. 7A, as well as in FIGS. 8A-11A, the fluid manipulation valve assembly 220 is illustrated without the elastically deformable cover such that the features of the substrate layer 222 can be seen without looking through the elastically deformable cover.

According to various embodiments, the currently closed valve 226 of the fluid manipulation valve assembly 220 is capable of being re-opened, and then re-closed. FIGS. 7B, 8B and 9B sequentially illustrate a procedure for re-opening the valve 226 starting from the first valve closing condition, according to various embodiments.

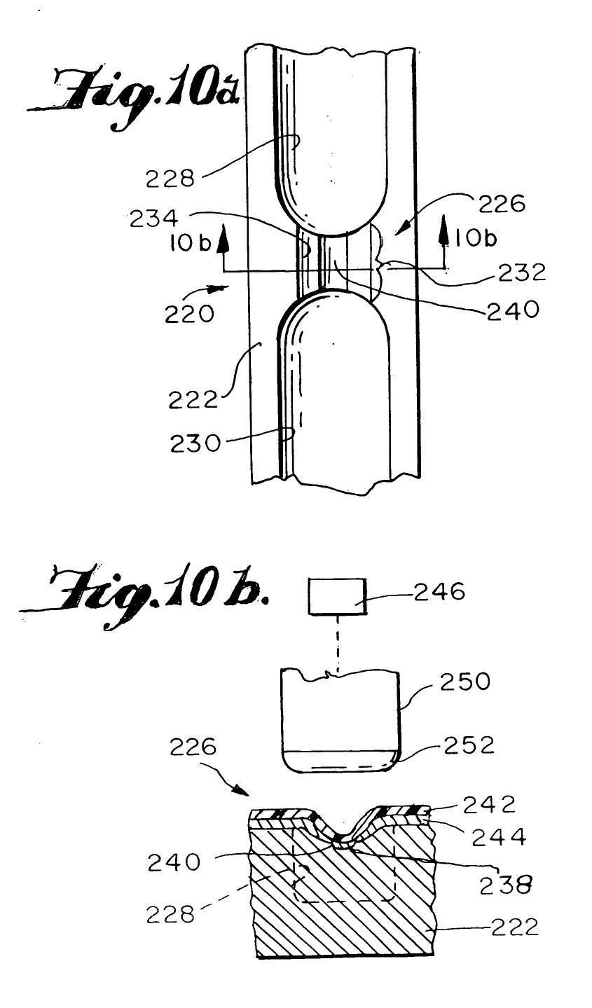

As can be seen in FIG. 8B, in a first re-opening step, the drive mechanism 246 can further actuate the first deformer 248 such that the contact surface 254 of the first deformer 248 deforms the cover layer 242 into the intermediate wall portion 232 of the substrate layer 222, thereby also displacing adhesive in a direction away from the first deformer 248. As a result, the intermediate wall 232 can be deformed by the deforming force of the first deformer 248 to form a deformation channel 240 in the substrate layer 222. With respect to FIG. 8B, the first deformer 248 can press the elastically deformable cover layer 242 through the adhesive layer 244 such that substantially none of the adhesive can be present between the cover layer 242 and the deformation channel 240. As a result, as discussed below with reference to FIG. 9B, when the first deformer 248 is removed from being in contact with the valve 226, the cover layer 242 can elastically rebound, forming a fluid communication opening 238.

FIG. 9B illustrates the second re-opening step which re-establishes the fluid communication between the recesses 228, 230. In the second re-opening step, the first deformer 248 is withdrawn from contacting the valve 226, thereby allowing the elastically deformable cover layer 242 to recover or rebound in a direction away from the deformation channel 240 formed in the intermediate wall 232. The inelastically deformable material of the intermediate wall 232 remains deformed, or remains deformed for a particular period of time, after the first deformer 248 is withdrawn. Upon recovering or rebounding, a portion of the elastically deformable cover layer 242 adjacent the deformation channel 240 of the intermediate wall 232, is spaced a set distance from the deformation channel 240 such that a fluid communication opening 238 can be formed. Thus, the fluid communication between the first and second recesses 228, 230 can be re-established.

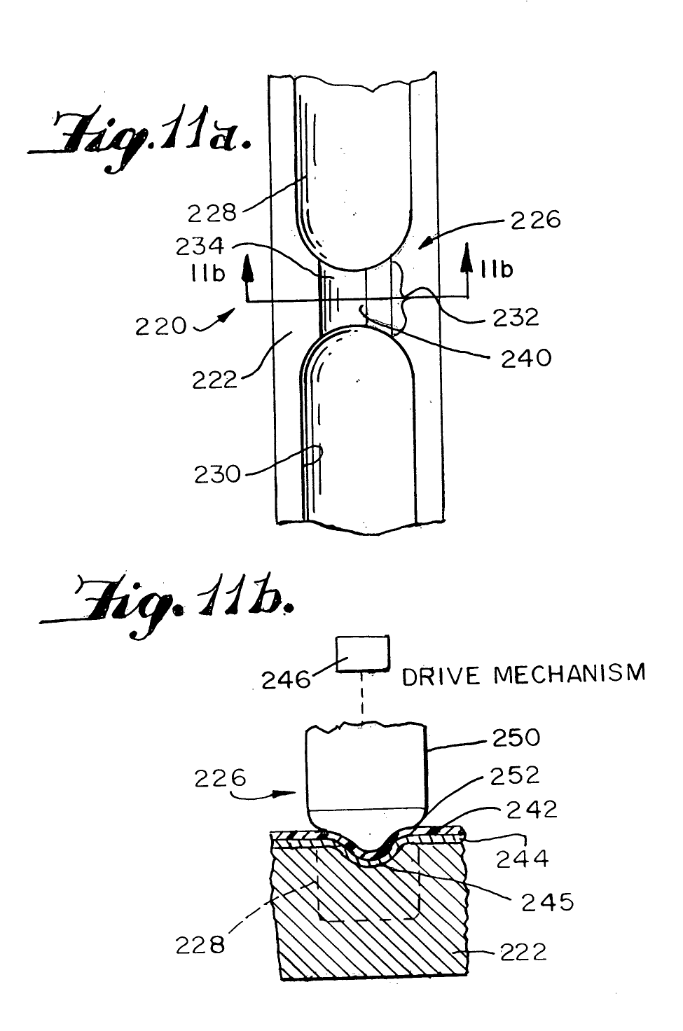

FIGS. 9B, 10B and 11B sequentially illustrate a procedure for re-closing the valve 226 starting from the condition that fluid communication between the first and second recesses 228, 230 has been re-established by way of the formation of the fluid communication opening 238. As can be seen in FIG. 10B, in a first re-closing step, the drive mechanism 246 can drive a second deformer 250 in a direction towards and into contact with the elastically deformable cover layer 242 of the open valve 226. The second deformer 250 can include a contact pad 252 or similar compliant device attached at an actuating end thereof.

FIG. 11B illustrates the second re-closing step which results in the fluid communication between the recesses 228, 230 being re-closed. In the second re-closing step, the drive mechanism 246 can force the contact pad 252 of the second deformer 250 into contact with the elastically deformable cover layer 242. When forcibly brought into contact with the cover layer 242, the contact pad 252 can mold into the shape of the depression formed by the cover layer 242, the adhesive layer 244 and the intermediate wall 232. As a result of the compliant or malleable characteristics of the pad 252, the material of the pad 252 can operate to manipulate the adhesive 245 of the adhesive layer 44 into the area of the fluid communication opening 238, thereby re-closing the valve 226.

The series of steps shown in FIGS. 6A-11A and FIGS. 11A-11B can be sequential or in any other order. For example, the valve 226 can be opened starting from an initially closed position, or the valve 226 can be closed from the initially open position shown in FIG. 10B.

The present teachings relate to the foregoing and other embodiments as will be apparent to those skilled in the art from consideration of the present specification and practice of the present teachings disclosed herein. It is intended that the present teachings be considered as exemplary only.

What is claimed is:

1. A microfluidic device comprising:
a first chamber adapted to retain one or more first components for a desired reaction;
a second chamber;
at least one second component retained in the second chamber, the at least one second component comprising one or more of an enzyme, a catalyst, an initiator, a promoter, and a cofactor, for the desired reaction; and
an openable communication between the first and second chambers.

2. The microfluidic device of claim 1, further including at least one first component retained in the first chamber.

3. The microfluidic device of claim 1, wherein the second component is a catalyst.

4. The microfluidic device of claim 3, wherein the catalyst contains magnesium.

5. The microfluidic device of claim 3, wherein the catalyst is an aqueous solution containing Mg²⁺ ions.

6. The microfluidic device of claim 1, wherein the second component is an initiator.

7. The microfluidic device of claim 1, wherein the second component is a promoter.

8. The microfluidic device of claim 1, wherein the second component is a cofactor.

9. The microfluidic device of claim 1, further comprising reactants for a nucleic acid sequencing or amplification reaction, the reactants being disposed in the first chamber.

10. The microfluidic device of claim 1, wherein the openable fluid communication comprises a valve.

11. The microfluidic device of claim 10, wherein the valve comprises a Zbig valve.

12. The microfluidic device of claim 10, wherein the valve comprises an adhesive material.

13. The microfluidic device of claim 10, wherein the valve comprises a recloseable valve.

14. The microfluidic device of claim 1, further comprising:
a third chamber; and
an openable fluid communication between the third chamber and at least one of the first and second chambers.

15. A method, comprising:
providing a microfluidic device comprising:
a first chamber;
at least one first component retained in the first chamber, the at least one first component comprising one or more reactant or reagent or component for the desired reaction; and
a second chamber;
at least one second component retained in the second chamber, the at least one second component comprising one or more of a catalyst, an initiator, a promoter, and a cofactor for a desired reaction; and
an openable communication between the first and second chambers;
opening the openable fluid communication between the first and second chambers;
at least one of combining and mixing the at least one first component with the at least one second component.

16. The method of claim 15, further comprising the step of heating the microfluidic device;

17. The method of claim 15, further comprising the step of opening the openable fluid communication between the first and second chambers.

18. The method of claim 15, wherein one or more of the at least one first component and the at least one second component comprise double-stranded DNA or double-stranded DNA fragments.

19. The method of claim 16, wherein heating the microfluidic device comprises heating of at least one of the first and second chambers to a temperature sufficient to denature the double-stranded DNA or the double-stranded DNA fragments.

20. The method of claim 16, further comprising:
cooling the microfluidic device to a temperature sufficient to allow single-stranded DNA or single-stranded DNA fragments to anneal to other single-stranded DNA or single-stranded DNA fragments.

21. The method of claim 20, wherein cooling the microfluidic device causes the mixture to undergo a nucleic acid, amplification, ligation, endonuclease, or sequencing reaction.

22. The method of claim 15, wherein the method further includes injecting a sample into the first chamber.

23. The method of claim 15, wherein at least one of the first chamber and the second chamber is at least partially pre-filled with a nucleic acid sequence amplification reaction component.

24. The method of claim 15, wherein at least one of the first chamber and the second chamber is pre-filled with a nucleic acid sequence amplification reaction component.

25. The method of claim 15, wherein at least one of the first chamber and the second chamber is at least partially pre-filled with a nucleic acid sequence detection reaction component.

26. The method of claim 15, wherein at least one of the first chamber and the second chamber is pre-filled with a nucleic acid sequence detection reaction component.

27. The method of claim 15, wherein at least one of the first chamber and the second chamber is at least partially pre-filled with a nucleic acid sequence restriction reactant component.

28. The method of claim 15, wherein at least one of the first chamber and the second chamber is pre-filled with a nucleic acid sequence restriction reaction component.

29. The method of claim 15, wherein causing the contents to combine comprises applying centripetal force to the first and second chambers.

30. The method of claim 15, wherein the first chamber retains a buffer, a polymerase, dNTPs, and at least one of a primer and a probe and the second chamber retains an aqueous solution of Mg²⁺ ions.

31. The method of claim 30, wherein at least some of the dNTPs are ddNTPs.

32. The method of claim 15, wherein the at least one second component comprises a catalyst.

33. The method of claim 15, wherein the at least one second component comprises an initiator.

34. The method of claim 15, wherein the at least one second component comprises a promoter.

35. The method of claim 15, wherein the at least one second component comprises a magnesium catalyst.

36. The method of claim 15, wherein the at least one second component comprises an enzyme.

37. The method of claim 15, wherein the at least one second component comprises a cofactor.

38. The method of claim 15, wherein at least one of the first and the second chambers are preheated.

39. The method of claim 15, wherein the at least one second component is a salt of magnesium that has been dried down in the second chamber.

40. The method of claim 15, wherein the at least one second component is a salt of magnesium.

41. The method of claim 15, wherein the at least one second component includes magnesium and glycerol.

42. The method of claim 15, wherein the at least one first component and the at least one second component are combined, the combined components are heated, and the heated combined components are mixed.

43. The method of claim 42, wherein the heated combined components are mixed by thermal mixing.

44. The method of claim 42, wherein the heated combined components are mixed by thermally-activated solutization.

45. The method of claim 42, wherein the heated combined components are mixed by vortexing.

46. The method of claim 42, wherein the heated combined components are mixed by sonication.

47. The method of claim 42, wherein the heated combined components are mixed by shaking.
